(19) United States
(12) Patent Application Publication
(10) Pub. No.: US 2021/0338730 A1
(43) Pub. Date: Nov. 4, 2021

(54) Marrow Infiltrating Lymphocytes With Increased Clonality and Uses Thereof

(71) Applicant: WINDMIL THERAPEUTICS, INC., Philadelphia, PA (US)

(72) Inventors: Kimberly A. Noonan, Philadelphia, PA (US); Eric R. Lutz, Philadelphia, PA (US)

(73) Assignee: WINDMIL THERAPEUTICS, INC., Philadelphia, PA (US)

(21) Appl. No.: 17/284,693

(22) PCT Filed: Nov. 1, 2019
(86) PCT No.: PCT/US2019/059426

§ 371 Date: Apr. 12, 2021

Related U.S. Application Data
(60) Provisional application No. 62/755,761, filed on Nov. 5, 2018.

(51) Int. Cl. A61K 35/17; G01N 33/50; C12N 5/0783

(52) U.S. Cl. CPC A61K 35/17; G01N 33/5011; G01N 33/505; C12N 5/0636; C12N 2501/2302; C12N 2501/515; C12N 2501/51; C12N 2500/02; G01N 2800/52

(57) ABSTRACT

The disclosure provides for compounds comprising cancer specific marrow infiltrating lymphocytes and methods for making and using the same.

### FIELD

The disclosure generally refers to marrow infiltrating lymphocytes (MILS) specific for treating cancer and methods of use thereof.

### BACKGROUND

Cancer is a leading cause of death and new therapies remain a clinical priority. Marrow infiltrating lymphocytes (MILS) are the product of activating and expanding bone marrow T cells. The bone marrow is a specialized niche in the immune system which is enriched for antigen experienced, central memory T cells. MILS have been shown to confer immunologically measurable clinical benefits in patients with cancer. The bone marrow microenvironment has also been shown to harbor tumor-antigen specific T cells in patients with solid tumors such as breast, liquid tumors, pancreatic and ovarian cancers, and the like, as well as hematological cancers such as multiple myeloma.

### SUMMARY OF THE INVENTION

It is an aspect of the invention to provide a composition comprising a population of marrow infiltrating lymphocytes comprising a plurality of TCRβs with affinities for a heterogeneous population of antigens.

It is a further aspect of the invention to provide a method for treating a subject having cancer with marrow infiltrating lymphocytes, the method comprising the steps of: (a) culturing a bone marrow sample obtained from the subject having cancer with an anti-CD3 antibody and an anti-CD28 antibody in a hypoxic environment to produce hypoxic-activated marrow infiltrating lymphocytes; (b) culturing the hypoxic-activated marrow infiltrating lymphocytes in a normoxic environment to produce the therapeutic activated marrow infiltrating lymphocytes comprising a plurality of TCRβs with affinities for a heterogeneous population of antigens; and (c) administering the therapeutic activated marrow infiltrating lymphocytes to the subject having cancer.

It is a further aspect of the invention to provide the method as described above, wherein the hypoxic environment has an oxygen content of about 1% to about 3% oxygen.

It is a further aspect of the invention to provide the method as described above, wherein the lymphocytes are cultured in the presence of IL-2.

It is a further aspect of the invention to provide the method as described above, wherein the culturing the hypoxic-activated marrow infiltrating lymphocytes in a normoxic environment is performed in the presence of IL-2.

It is a further aspect of the invention to provide the method as described above, wherein the bone marrow sample is cultured in the hypoxic environment for about 24 hours.

It is a further aspect of the invention to provide the method as described above, wherein the bone marrow sample is cultured in the hypoxic environment for about 2 days.

It is a further aspect of the invention to provide the method as described above, wherein the bone marrow sample is cultured in the hypoxic environment for about 3 days.

It is a further aspect of the invention to provide the method as described above, wherein the bone marrow sample is cultured in the hypoxic environment for about 2 to about 5 days.

It is a further aspect of the invention to provide the method as described above, wherein the hypoxic environment is about 1% to about 2% oxygen.

It is a further aspect of the invention to provide the method as described above, wherein the hypoxic-activated marrow infiltrating lymphocytes are cultured in the normoxic environment for about 2 to about 12 days.

It is a further aspect of the invention to provide the method as described above, wherein the hypoxic-activated marrow infiltrating lymphocytes are cultured in the normoxic environment for about 6 days.

It is a further aspect of the invention to provide the method as described above, wherein the hypoxic-activated marrow infiltrating lymphocytes are cultured in the normoxic environment for about 9 days.

It is a further aspect of the invention to provide the method as described above, further comprising the step of removing a bone marrow sample from a subject having cancer prior to step (a).

It is a further aspect of the invention to provide the method as described above, wherein the anti-CD3 antibody and the anti-CD28 antibody are bound on a bead.

It is a further aspect of the invention to provide the method as described above, wherein the cancer is one or more of the cancers described herein.

It is a further aspect of the invention to provide the method as described above, wherein the cancer is selected from the group consisting of myeloma; lung cancer; prostate cancer; breast cancer; colon cancer; skin cancer; sarcomas and carcinomas selected from the group consisting of fibrosarcoma, myxosarcoma, liposarcoma, chondrosarcoma, and osteosarcoma; synovioma; mesothelioma; Ewing's tumor; leiomyosarcoma; rhabdomyosarcoma; lymphoid malignancy; pancreatic cancer; ovarian cancer; hepatocellular carcinoma; squamous cell carcinoma; basal cell carcinoma; adenocarcinoma; sweat gland carcinoma; medullary thyroid carcinoma; papillary thyroid carcinoma; pheochromocytomas sebaceous gland carcinoma; papillary carcinoma; papillary adenocarcinomas; medullary carcinoma; bronchogenic carcinoma; renal cell carcinoma; hepatoma; bile duct carcinoma; choriocarcinoma; Wilms' tumor; cervical cancer; testicular tumor; seminoma; bladder carcinoma; melanoma; CNS tumors selected from the group consisting of glioma, brainstem glioma, mixed gliomas, glioblastoma astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, and brain metastases, and combinations thereof.

It is a further aspect of the invention to provide the method as described above, wherein the cancer is a hematological or hematogenous cancer selected from the group consisting of leukemia, acute leukemias, acute lymphocytic leukemia, acute myelocytic leukemia, acute myelogenous leukemia, myeloblastic, promyelocytic, myelomonocytic, monocytic erythroleukemia, chronic leukemias, chronic myelocytic (granulocytic) leukemia, chronic myelogenous leukemia, and chronic lymphocytic leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, myelodysplasia, and combinations thereof.

It is a further aspect of the invention to provide a method for treating a subject having cancer with therapeutic activated marrow infiltrating lymphocytes comprising a population of marrow infiltrating lymphocytes comprising a plurality of TCRβs with affinities for a heterogeneous population of antigens, the method comprising the steps of: (a) culturing a bone marrow sample obtained from the subject having cancer with anti-CD3/anti-CD28 beads in a hypoxic environment of about 1% to about 2% oxygen for about 2 to about 5 days to produce hypoxic-activated marrow infiltrating lymphocytes comprising the population of marrow infiltrating lymphocytes comprising a plurality of TCRβs with affinities for a heterogeneous population of antigens; (b) culturing the hypoxic-activated marrow infiltrating lymphocytes in a normoxic environment of about 21% oxygen for about 2 to about 12 days in the presence of IL-2 to produce the therapeutic activated marrow infiltrating lymphocytes; and (c) administering the therapeutic activated marrow infiltrating lymphocytes to the subject having cancer.

It is a further aspect of the present invention to provide a method of treating cancer in a subject, the method comprising administering a pharmaceutical composition comprising the composition as described above to the subject.

It is a further aspect of the invention to provide the method as described above, wherein the population of marrow infiltrating lymphocytes is obtained from a subject having cancer.

It is a further aspect of the invention to provide the method as described above, wherein the cancer specific marrow infiltrating lymphocyte is autologous to the subject being treated.

It is a further aspect of the invention to provide the method as described above, wherein the cancer specific marrow infiltrating lymphocyte is allogeneic to the subject being treated.

It is a further aspect of the invention to provide the method as described above, wherein the marrow infiltrating lymphocyte is hypoxic activated.

It is a further aspect of the invention to provide the method as described above, wherein the marrow infiltrating lymphocyte is hypoxic activated and normoxic activated.

It is a further aspect of the invention to provide the method as described above, wherein the pharmaceutical composition is administered by parenteral administration, intraperitoneal or intramuscular administration.

It is a further aspect of the invention to provide the method as described above, wherein the about 75% to about 100% of marrow infiltrating lymphocytes administered to the subject express CD3.

It is a further aspect of the invention to provide the method as described above, wherein the about 80% to about 100% of marrow infiltrating lymphocytes administered to the subject express CD3.

It is a further aspect of the invention to provide the method as described above, wherein the about 85% to about 100% of marrow infiltrating lymphocytes administered to the subject express CD3.

It is a further aspect of the invention to provide the method as described above, wherein the about 90% to about 100% of marrow infiltrating lymphocytes administered to the subject express CD3.

It is a further aspect of the invention to provide the method as described above, wherein the ratio of CD4⁺:CD8⁺ T cells present in the composition or MILS administered to the subject is about 2:1.

It is a further aspect of the invention to provide a composition comprising a population of hypoxic-activated marrow infiltrating lymphocytes isolated from a patient with cancer comprising a population of marrow infiltrating lymphocytes comprising a plurality of TCRβs with affinities for a heterogeneous population of antigens, wherein about 75% to about 100% of the population of the hypoxic activated marrow infiltrating lymphocytes expresses CD3.

It is a further aspect of the invention to provide the composition as described above, wherein about 80% to about 100% of the population of the hypoxic activated marrow infiltrating lymphocytes expresses CD3.

It is a further aspect of the invention to provide the composition as described above, wherein about 85% to about 100% of the population of the hypoxic activated marrow infiltrating lymphocytes expresses CD3.

It is a further aspect of the invention to provide the composition as described above, wherein about 90% to about 100% of the population of the hypoxic activated marrow infiltrating lymphocytes expresses CD3.

It is a further aspect of the invention to provide the composition as described above, wherein the ratio of CD4⁺:CD8⁺ T cells present in the composition is about 2:1.

It is a further aspect of the invention to provide the composition as described above, wherein the cell population is obtainable from a bone marrow sample obtained from a subjecting having cancer by: (a) culturing the bone marrow sample with an anti-CD3 antibody and an anti-CD28 antibody in a hypoxic environment of about 1% to about 3% oxygen to produce activated marrow infiltrating lymphocytes; and (b) culturing the activated marrow infiltrating lymphocytes in a normoxic environment in the presence of IL-2 to produce the composition.

It is a further aspect of the invention to provide the composition as described above, wherein the MILS are cancer specific.

It is a further aspect of the invention to provide a method of predicting which patients are responding to infusion with MILS comprising: A) isolating MILS from patients post-infusion; B) determining the clonality of post-infusion isolated MILS; C) continuing successful treatment with MILS if clonality is increased as compared to the clonality in pre-treatment bone marrow from the same patients.

It is a further aspect of the invention to provide a method of predicting which patients are responding to infusion with MILS comprising: A) measuring clonality of baseline pre-infusion bone marrow; B) measuring clonality of post-infusion bone marrow; C) continuing successful treatment with MILS if clonality increases post-infusion as compared to pre-infusion bone marrow.

It is a further aspect of the invention to provide a method of predicting which patients are responding to infusion with MILS comprising: A) measuring clonality of baseline pre-infusion blood; B) measuring clonality of post-infusion blood; C) continuing successful treatment with MILS if clonality increases post-infusion as compared to pre-infusion blood.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

FIG. 1: FIG. 1 shows the Phase I clinical trial design and sample collection schedule. The circled stars represent the time points at which the ImmunoSEQ procedure was performed on the collected bone marrow and blood samples.

FIG. 2: FIG. 2 shows IFNγ capture isolation of tumor-antigen-specific MILS.

FIG. 3: FIG. 3 shows the clonal frequency distribution. The method for calculating is described in Example 3.

FIG. 4: FIG. 4 shows a summary of the immunoSEQ data. The sequencing was successful and all samples passed quality control. The antigen-specific sorted T cells samples had greater than 100 T cells, and greater than 1000 T cells on average. Productive templates equal the total number of T cells in the specimen. Productive rearrangements are equal to the total number of unique CDR3 sequences in the specimen.

FIGS. 5A and 5B: FIGS. 5A and 5B show the tracking of antigen-specific TCRβ CDR3s and T cells in MILS. The percentage of unique TCRβ CDR3s (FIG. 5A) and T cells (FIG. 5B) identified as being tumor antigen-specific are reported for each patient's MILS product. Approximately 0.3-1.4% of unique TCRβ CDR3 sequences and 2-15% of T cells in MILS are estimated to be tumor antigen-specific.

FIG. 6: FIG. 6 shows that the frequency of tumor-antigen-specific T cells in the MILS product does not correlate with clinical antitumor response. The percentage of unique TCRβ CDR3s (left) and T cells (right) identified as being tumor antigen-specific for each patient's MILS product were compared between clinical responders “CRs” and progressed disease “PDs” using Wilcoxon rank sum tests. P values were >0.05.

FIG. 7: FIG. 7 shows the tracking of the frequency of MILS in bone marrow and blood. The cumulative frequencies of MILS are shown tracked in pre and post-infusion BM and PBMC. CRs are indicated with solid lines and PDs with dotted lines. Similar frequencies and kinetics were observed in BM and PBMC. CRs start with the lowest pre-treatment frequency of MILS and CD8-Ag-spec-MILS. Larger increases in MILS frequencies were observed in CRs compared to PDs.

FIG. 8: FIG. 8 shows that persistent increases in MIL frequency correlate with clinical antitumor response. Post-treatment fold-change in the frequency of MILS in BM and PBMC are shown for each patient. CRs are indicated with solid lines and PDs with dotted lines. CRs have more persistent increases in MILS frequency. At day 360, change in MILS frequency perfectly segregates CRs and PDs (red boxes).

FIGS. 9 and 10: FIGS. 9 and 10 show that the tumor antigen-specific T-cell repertoires in MILS are highly polyclonal. The clonality of pre-treatment BM (“pre-BM”), MILS, CD4+, and CD8+ tumor antigen-specific MILS were compared between CRs and PDs using t-tests (*p<0.05) (see FIG. 9). For all 6 patients, MILS were more polyclonal than pre-BM. Pre-BM was more polyclonal in CRs compared to PDs. TCRβ V gene family gene usage is shown for CD4+ and CD8+ tumor antigen specific MILS compared to all other MILS (See FIG. 10). There were no clear differences in gene usage between antigen specific MILS and other MILS.

FIG. 11: FIG. 11 shows tracking of the clonality in bone marrow and blood. Clonality was tracked in pre and post-treatment BM (left) and PBMC (right). CRs are indicated with solid lines and PDs with dotted lines. Trends in BM and PBMC were consistent. The clonality increased in all 3 CRs and only 1/3 PDs. Pre-treatment clonality was lower in CR subjects (red boxes).

FIG. 12: FIG. 12 shows that the persistent increases in clonality correlate with clinical antitumor response. The post-treatment fold-change in clonality in BM (left) and PBMC (right) is shown for each patient. CRs are indicated with solid lines and PDs with dotted lines. All three CRs maintained an increase in clonality out to day 360 (red boxes), while PDs returned to or dropped below baseline.

FIG. 13: FIG. 13 shows tracking of expanding T-cell clonotypes in bone marrow and blood. In the top panel, it is shown that most expanding clones are CD8-Ag-spec-MILS. In the bottom panel, it is shown that very few expanding clones are CD8-Ag-spec-MILS. Frequencies of clonotypes in pre-treatment BM (X-axis on both panels) vs in BM 60 days post-treatment (Y-axis on both panels) for two patients (one CR and one PD). Significantly-expanded clonotypes (DeWitt et al. J. Virol. 2015; 89(8):4517-26.) are shaded yellow. CD8+ tumor antigen-specific clonotypes are outlined.

FIG. 14: FIG. 14 shows tracking of expanding tumor antigen-specific T-cell clonotypes that are undetectable in pre-treatment bone marrow and blood. The number of expanded tumor antigen-specific clonotypes that were undetectable pre-treatment are shown for each patient at each post-treatment time point. CRs are indicated with green lines and PDs with black lines. These are likely rare tumor antigen-specific clonotypes that expand following treatment. Higher numbers of these clonotypes are seen in 2/3 CRs.

### DETAILED DESCRIPTION

MILS are an autologous T-cell product expanded from bone marrow (BM) being developed as a novel cell therapy for both hematological and solid malignancies. In a Phase I trial evaluating MILS in patients with advanced multiple myeloma, 6 (27.3%) of 22 patients achieved a complete remission (CR). Immune analyses demonstrated that the establishment of persistent tumor antigen-specific T cells in BM correlated with improved clinical responses (Noonan K. A., Huff C. A., Davis J., Lemas M. V., Fiorino S., Bitzan J., Ferguson A., Emerling A., Borrello I. Adoptive transfer of activated marrow-infiltrating lymphocytes induces measurable antitumor immunity in the bone marrow in multiple myeloma. Sci. Transl. Med. 2015; 7: 288ra78, which is incorporated by reference).

T cell clonotypes were identified within MILS, including the subset that specifically recognize tumor antigens; to track and compare their frequencies in blood and BM before and after infusion; and to compare T cell repertoire characteristics, such as clonality, between clinical responders and non-responders.

The TCRβ CDR3 was sequenced using Adaptive Biotechnologies' immunoSEQ Assay and used to identify and track MILS T cell clonotypes. The immunoSEQ assay was used on 11 specimens (unsorted MILS, IFNγ-capture-sorted tumor antigen-specific CD4+ and CD8+ T cells, and blood and BM collected pre-treatment and 60, 180 and 360 days post-infusion) from 6 patients (3 clinical responders who achieved a CR and 3 non-responders whose disease progressed) from the Phase I study.

When cumulative frequencies of MILS T-cell clonotypes were tracked in BM and blood, there were significant differences between responders and non-responders. Responders had a lower frequency of clonotypes at baseline but showed larger and more persistent increases in the frequency of clonotypes in both BM and blood. At day 360, fold-change from baseline in the frequency of MILS in both compartments segregated responders from non-responders.

In general, T-cell repertoires in MILS were highly polyclonal and no specific TCRβ variable genes were enriched in tumor antigen-specific T cells suggesting that multiple antigens are targeted. In all 6 patients, MILS were more polyclonal than pre-expanded BM. However, starting repertoires were more polyclonal in responders, and responders had larger and more persistent post-infusion increases in clonality. At day 360, all 3 responders maintained an increase in clonality whereas clonality returned to baseline or lower in all 3 non-responders.

These data provide a 1st look at the repertoire of T cell clonotypes in MILS and how the repertoire evolves after treatment at the clonal level. The data also demonstrate the highly polyclonal nature of tumor antigen-specific T cells within MILS, which could provide an advantage against heterogeneous tumors.

As used herein and unless otherwise indicated, the term “about” is intended to mean±5% of the value it modifies. Thus, about 100 means 95 to 105. Additionally, the term “about” modifies a term in a series of terms, such as “about 1, 2, 3, 4, or 5” it should be understood that the term “about” modifies each of the members of the list, such that “about 1, 2, 3, 4, or 5” can be understood to mean “about 1, about 2, about 3, about 4, or about 5.” The same is true for a list that is modified by the term “at least” or other quantifying modifier, such as, but not limited to, “less than,” “greater than,” and the like.

As used herein and in the appended claims, the singular forms “a”, “an” and “the” include plural reference unless the context clearly dictates otherwise.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the terms “treat,” “treated,” or “treating” mean both therapeutic treatment wherein the object is to slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Thus, “treatment of cancer” or “treating cancer” means an activity that alleviates or ameliorates any of the primary phenomena or secondary symptoms associated with the cancer or any other condition described herein. In some embodiments, the cancer that is being treated is one of the cancers recited herein.

As used herein, the term “subject” can be used interchangeably with the term “patient”. The subject can be a mammal, such as a dog, cat, monkey, horse, cow, and the like. In some embodiments, the subject is a human. In some embodiments, the subject has been diagnosed with cancer. In some embodiments, the subject is believed to have cancer. In some embodiments, the subject is suspected of having cancer.

As used herein, the term “express” as it refers to a cell surface receptor, such as, but not limited to, CD3, CD4, and CD8, can also be referred to as the cell being positive for that marker. For example a cell that expresses CD3 can also be referred to as CD3 positive (CD3⁺).

The term “cancer” as used herein is defined as disease characterized by the rapid and uncontrolled growth of aberrant cells. Cancer cells can spread locally or through the bloodstream and lymphatic system to other parts of the body. Examples of cancers that can be treated with the MILS provided for herein, include, but are not limited to, myeloma, lung cancer, prostate cancer, breast cancer, colon cancer, skin cancer, sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, lymphoid malignancy, pancreatic cancer, ovarian cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases) and the like.

The compositions and methods provided herein can also be used for hematological cancers. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein, “marrow infiltrating lymphocytes” or “MILS” are a subpopulation of immune cells and are described for example in, U.S. Pat. No. 9,687,510, which is hereby incorporated by reference in its entirety. MILS significantly differ from peripheral lymphocytes (PBLs). For example, MILS are more easily expanded, upregulate activation markers to a greater extent than PBLs, maintain more of a skewed Vβ repertoire, traffic to the bone marrow, and most importantly, possess significantly greater tumor specificity. MILS anti-myeloma immunity correlates directly with clinical response; however, no in vivo T cell expansion or persistent clinical response has previously been observed following infusion. In some embodiments, MILS can be activated, for example, by incubating them with anti-CD3/anti-CD-28 beads and under hypoxic conditions, as described herein. In some embodiments, growing MILS under hypoxic conditions is also described in U.S. Pat. No. 9,687,510, and International Application No. WO2016/037054, both of which are incorporated by reference herein in their entirety.

In some embodiments, methods to prepare MILS may comprise removing cells from the bone marrow, lymphocytes, and/or marrow infiltrating lymphocytes from the subject; incubating the cells in a hypoxic environment, thereby producing activated MILS. In some embodiments, the subject has cancer. The cells can also be activated in the presence of anti-CD3/anti-CD28 antibodies and cytokines as described herein.

The hypoxic environment may comprise less than about 21% oxygen, such as less than about 20%, 19%, 18%, 17%, 16%, 15%, 14%, 13%, 12%, 11%, 10%, 9%, 8%, 7%, 6%, 5%, 4%, or less than about 3% oxygen. For example, the hypoxic environment may comprise about 0% oxygen to about 20% oxygen, such as about 0% oxygen to about 19% oxygen, about 0% oxygen to about 18% oxygen, about 0% oxygen to about 17% oxygen, about 0% oxygen to about 16% oxygen, about 0% oxygen to about 15% oxygen, about 0% oxygen to about 14% oxygen, about 0% oxygen to about 13% oxygen, about 0% oxygen to about 12% oxygen, about 0% oxygen to about 11% oxygen, about 0% oxygen to about 10% oxygen, about 0% oxygen to about 9% oxygen, about 0% oxygen to about 8% oxygen, about 0% oxygen to about 7% oxygen, about 0% oxygen to about 6% oxygen, about 0% oxygen to about 5% oxygen, about 0% oxygen to about 4% oxygen, or about 0% oxygen to about 3% oxygen. In some embodiments, the hypoxic environment comprises about 1% to about 7% oxygen. In some embodiments, the hypoxic environment is about 1% to about 2% oxygen. In some embodiments, the hypoxic environment is about 0.5% to about 1.5% oxygen. In some embodiments, the hypoxic environment is about 0.5% to about 2% oxygen. The hypoxic environment may comprise about 20%, 19%, 18%, 17%, 16%, 15%, 14%, 13%, 12%, 11%, 10%, 9%, 8%, 7%, 6%, 5%, 4%, 3%, 2%, 1%, or about 0% oxygen. In some embodiments, the hypoxic environment comprises about 7%, 6%, 5%, 4%, 3%, 2%, or 1% oxygen.

Incubating MILS in a hypoxic environment may comprise incubating the MILS, e.g., in tissue culture medium, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 11 days, 12 days, 13 days, or even at least about 14 days. Incubating may comprise incubating the MILS for about 1 hour to about 30 days, such as about 1 day to about 20 days, about 1 day to about 14 days, or about 1 day to about 12 days. In some embodiments, incubating MILs in a hypoxic environment comprises incubating the MILs in a hypoxic environment for about 2 days to about 5 days. The method may comprise incubating MILS in a hypoxic environment for about 1 day, 2 days, 3 days, 4 days, 5 days, 6 days, 7 days, 8 day, 9 days, 10 days, 11 days, 12 days, 13 days, or 14 days. In some embodiments, the method comprises incubating the MILS in a hypoxic environment for about 3 days. In some embodiments, the method comprises incubating the MILS in a hypoxic environment for about 2 days to about 4 days. In some embodiments, the method comprises incubating the MILS in a hypoxic environment for about 3 days to about 4 days.

In some embodiments, hypoxic-activated MILS are then cultured in a normoxic environment to produce the therapeutic activated marrow infiltrating lymphocytes. In some embodiments, the normoxic environment may comprise at least about 21% oxygen. In some embodiments, the normoxic environment may comprise about, such as about 10% oxygen to about 30% oxygen, about 15% oxygen to about 25% oxygen, about 18% oxygen to about 24% oxygen, about 19% oxygen to about 23% oxygen, or about 20% oxygen to about 22% oxygen. In some embodiments, the normoxic environment comprises about 21% oxygen.

In some embodiments, the MILS are cultured in the presence of IL-2 or other cytokines. In some embodiments, the MILS are cultured in normoxic conditions in the presence of IL-2. In some embodiments, the other cytokines can be IL-7, IL-15, IL-9, IL-21, or any combination thereof. In some embodiments, the MILS can be cultured in cell culture medium that comprises one or more cytokines, e.g., such as IL-2, IL-7, and/or IL-15, or any suitable combination thereof. Illustrative examples of suitable concentrations of each cytokine or the total concentration of cytokines includes, but is not limited to, about 25 IU/mL, about 50 IU/mL, about 75 IU/mL, about 100 IU/mL, about 125 IU/mL, about 150 IU/mL, about 175 IU/mL, about 200 IU/mL, about 250 IU/mL, about 300 IU/mL, about 350 IU/mL, about 400 IU/mL, about 450 IU/mL, or about 500 IU/mL or any intervening amount of cytokine thereof. In some embodiments, the cells are cultured in about 100 IU/mL of each of, or in total of, IL-2, IL-1, and/or IL-15, or any combination thereof. In some embodiments, the cell culture medium comprises about 250 IU/mL of each of, or in total of, IL-2, IL-1, and/or IL-15, or any combination thereof.

Incubating MILS in a normoxic environment may comprise incubating the MILS, for at least about 1 hour, such as at least about 12 hours, 18 hours, 24 hours, 30 hours, 36 hours, 42 hours, 48 hours, 60 hours, 3 days, 4 days, 5 days, 6 days, 7 days, 8 days, 9 days, 10 days, 11 days, 12 days, 13 days, or even at least about 14 days. Incubating may comprise incubating the MILS for about 1 hour to about 30 days, such as about 1 day to about 20 days, about 1 day to about 14 days, about 1 day to about 12 days, or about 2 days to about 12 days.

In some embodiments, the MILS are obtained by extracting a bone marrow sample from a subject and culturing/incubating the cells as described herein. In some embodiments, the bone marrow sample is centrifuged to remove red blood cells. In some embodiments, the bone marrow sample is not subject to apheresis. In some embodiments, the bone marrow sample does not comprise peripheral blood lymphocytes (“PBLs”) or the bone marrow sample is substantially free of PBLs. These methods select for cells that are not the same as what have become to be known as TILs. Thus, a MIL is not a TIL. TILs can be selected by known methods to one of skill in the art and can be transfected or infected with the nucleic acid molecules described herein such that the TILs can express the chimeric transmembrane protein described herein. In some embodiments, the bone marrow sample contains less than 10%, 9%, 8%, 7%, 6%, 5%, 4%, 3%, 2%, 1% PBLs as compared to the total of MILS. In some embodiments, the sample is free of PBLs.

In some embodiments, the cells are also activated by culturing with antibodies to CD3 and CD28. This can be performed, for example by incubating the cells with anti-CD3/anti-CD28 beads that are commercially available or that can be made by one of skill in the art. The cells can then be plated in a plate, flask, or bag. Hypoxic conditions can be achieved by flushing either the hypoxic chamber or cell culture bag for 3 minutes with a 95% Nitrogen and 5% CO2 gas mixture. This can lead to, for example, 1-2% or less 02 gas in the receptacle. Examples of such beads and methods of stimulation can be found, for example, in U.S. Pat. Nos. 6,352,694, 6,534,055, 6,692,964, 6,797,514, 6,867,041, 6,905,874, each of which are incorporated by reference in its entirety. Alternatives to beads are engineered cells, such as K562 cells, that can be used to stimulate the MILS. Such methods can be found in, for example, U.S. Pat. Nos. 8,637,307 and 7,638,325, each of which are incorporated by reference in its entirety. Cells can also be stimulated using other methods, such as those described in U.S. Pat. No. 8,383,099, which is incorporated by reference in its entirety.

In some embodiments, activated MILS and/or therapeutic activated MILS are administered to a subject having, or suspected of having, cancer. In some embodiments, hypoxic-activated MILS and/or therapeutic activated MILS are produced from a bone marrow sample from a subject having or suspected of having cancer, then administering to the same subject to treat cancer. In some embodiments, the MILS are allogeneic to the subject.

In some embodiments, methods are provided for inducing the expansion of tumor antigen-specific T cells that are at very low frequencies and undetectable by immunoSEQ prior to treatment. In some embodiments, the methods comprising administering MILS with a high degree of clonality to the subject, thereby increasing antigen-specific T cells that are at very low frequencies and undetectable by immunoSEQ prior to treatment.

In some embodiments, the MILS can be administered in a pharmaceutical preparation or pharmaceutical composition. Pharmaceutical compositions comprising the cancer specific MILS may further comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions can be formulated for parenteral administration, e.g., intravascular (intravenous or intraarterial), intraperitoneal or intramuscular administration. In some embodiments, the MILS and/or compositions are administered by parenteral administration, e.g., intravascular (intravenous or intraarterial), intraperitoneal or intramuscular administration. The compositions can also be administered directly into the tumor. In some embodiments, the compositions are administered intravenously.

In some embodiments, compositions, whether they be solutions, suspensions or other like form, may include one or more of the following: DMSO, sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the subject can be pre-conditions with cyclophosphamide with or without fludarabine. One such example is provided for in U.S. Pat. No. 9,855,298, which is hereby incorporated by reference. Another non-limiting example is administering fludarabine (30 mg/m2 intravenous daily for 4 days) and cyclophosphamide (500 mg/m2 intravenous daily for 2 days starting with the first dose of fludarabine). After administration, the MILS can be administered 2 to 14 days after completion of the fludarabine. In some embodiments, the cyclophosphamide is administered or 2-3 days at a dose of about 500 to about 600 mg/m2).

In some embodiments, the pharmaceutical composition that is administered comprises cancer specific MILS as provided for herein. A composition of such MILS is also provided for herein. In some embodiments, the cancer specific MILS are hypoxic activated. In some embodiments, the cancer specific MILS are hypoxic activated/normoxic activated MILS. A cancer specific MIL is a MIL that can specifically target the cancer in a subject.

In some embodiments, the composition comprises a population of cancer specific MILS that are CD3 positive. In some embodiments, at least about, or at least, 40% of the MILS are CD3 positive. In some embodiments, about, or at least, 45%, 50%, 55%, 60%, 65%, 70%, 75%, 80%, 85%, 86%, 87%, 88%, or 89% of MILS are CD3 positive. In some embodiments, at least, or about, 80% of the MILS are CD3 positive. In some embodiments, about 40% to about 100% of the MILS are CD3 positive. In some embodiments, about 45% to about 100%, about 50% to about 100%, about 55% to about 100%, about 60% to about 100%, about 65% to about 100%, about 70% to about 100%, about 75% to about 100%, about 80% to about 100%, about 85% to about 100%, about 86% to about 100%, about 87% to about 100%, about 88% to about 100%, or about 90% to about 100% of the MILS are CD3 positive (express CD3).

In some embodiments, the composition comprises either a population of MILS that do not express CD3, or a population of MILS that expresses low levels of CD3, for example, relative to the expression level of MILS from the population of MILS that express CD3.

In some embodiments, the composition comprises a population of MILS that expresses interferon gamma (“IFNγ”), i.e., wherein each cell in the population of MILS that expresses IFNγ is a marrow infiltrating lymphocyte that expresses IFNγ, e.g., as detected by flow cytometry. For example, at least about 2% of the cells in the composition may be MILS that express IFNγ, or at least about 2%, 3%, 4%, 5%, 6%, 7%, 8%, 9%, 10%, 11%, 12%, 13%, 14%, 15%, 16%, 17%, or even at least about 18% of the MILS express IFNγ. In some embodiments, about 2% to about 100% of the MILS express IFNγ, such as about 2% to about 100%, about 3% to about 100%, about 4% to about 100%, about 5% to about 100%, about 6% to about 100%, about 7% to about 100%, about 8% to about 100%, about 9% to about 100%, about 10% to about 100%, about 11% to about 100%, about 12% to about 100%, about 13% to about 100%, about 14% to about 100%, about 15% to about 100%, about 16% to about 100%, about 17% to about 100%, or even about 18% to about 100% of the MILS. In some embodiments, the composition comprises either a population of MILS that do not express IFNγ, e.g., as detected by flow cytometry, or a population of MILS that expresses low levels of IFNγ, i.e., relative to the expression level of MILS from the population of MILS that express IFNγ.

In some embodiments, the composition comprises a population of MILS that expresses CXCR4. For example, at least about 98% of the MILS express CXCR4, such as at least about 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, or even at least about 99.7% of the MILS. In some embodiments, about 98% to about 100% may be MILS that express CXCR4, such as at least about 98.1% to about 100%, about 98.2% to about 100%, about 98.3% to about 100%, about 98.4% to about 100%, about 98.5% to about 100%, about 98.6% to about 100%, about 98.7% to about 100%, about 98.8% to about 100%, about 98.9% to about 100%, about 99.0% to about 100%, about 99.1% to about 100%, about 99.2% to about 100%, about 99.3% to about 100%, about 99.4% to about 100%, about 99.5% to about 100%, about 99.6% to about 100%, or even about 99.7% to about 100% of the MILS in the composition. In some embodiments, the composition comprises either a population of MILS that do not express CXCR4, e.g., as detected by flow cytometry, or a population of MILS that expresses low levels of CXCR4, i.e., relative to the expression level of MILS from the population of MILS that express CXCR4.

The population of MILS that expresses CD4 may comprise a plurality of MILS that expresses 4-1BB. For example, at least about 21% of the cells in the composition may be MILS from the plurality of MILS that expresses 4-1BB, such as at least about 22%, 23%, 24%, 25%, 26%, 27%, 28%, 29%, 30%, 31%, 32%, 33%, 34%, 35%, 36%, 37%, 38%, 39%, 40%, 41%, 42%, or even at least about 43% of the cells in the composition. In some embodiments, about 21% to about 100% of the cells in the composition may be MILS from the plurality of MILS that expresses 4-1BB, such as about 22% to about 100%, about 23% to about 100%, about 24% to about 100%, about 25% to about 100%, about 26% to about 100%, about 27% to about 100%, about 28% to about 100%, about 29% to about 100%, about 30% to about 100%, about 31% to about 100%, about 32% to about 100%, about 33% to about 100%, about 34% to about 100%, about 35% to about 100%, about 36% to about 100%, about 37% to about 100%, about 38% to about 100%, about 39% to about 100%, about 40% to about 100%, about 41% to about 100%, about 42% to about 100%, or even about 43% to about 100% of the cells in the composition.

The composition may comprise a population of MILS that expresses CD8. The population of MILS that expresses CD8 may comprise a plurality of MILS that expresses CXCR4.

The population of MILS that expresses CD8 may comprise a plurality of MILS that expresses 4-1BB. For example, at least about 21% of the cells in the composition may be MILS from the plurality of MILS that expresses 4-1BB, such as at least about 8%, 9%, 10%, 11%, 12%, 13%, 14%, 15%, 16%, 17%, 18%, 19%, 20% or even at least about 21% of the cells in the composition. In some embodiments, about 2% to about 100% of the cells in the composition may be MILS from the plurality of MILS that expresses 4-1BB, such as about 8% to about 100%, about 9% to about 100%, about 10% to about 100%, about 11% to about 100%, about 12% to about 100%, about 13% to about 100%, about 14% to about 100%, about 15% to about 100%, about 16% to about 100%, about 17% to about 100%, about 18% to about 100%, about 19% to about 100%, about 20% to about 100%, or even about 21% to about 100% of the cells in the composition.

In some embodiments, the composition comprises a population of MILS that expresses 4-1BB. For example, at least about 21% of the cells in the composition may be MILS from the population of MILS that expresses 4-1BB, such as at least about 22%, 23%, 24%, 25%, 26%, 27%, 28%, 29%, 30%, 31%, 32%, 33%, 34%, 35%, 36%, 37%, 38%, 39%, 40%, 41%, 42%, or even at least about 43% of the cells in the composition. In some embodiments, about 21% to 100% of the cells in the composition may be MILS from the population of MILS that expresses 4-1BB, such as about 22% to about 100%, about 23% to about 100%, about 24% to about 100%, about 25% to about 100%, about 26% to about 100%, about 27% to about 100%, about 28% to about 100%, about 29% to about 100%, about 30% to about 100%, about 31% to about 100%, about 32% to about 100%, about 33% to about 100%, about 34% to about 100%, about 35% to about 100%, about 36% to about 100%, about 37% to about 100%, about 38% to about 100%, about 39% to about 100%, about 40% to about 100%, about 41% to about 100%, about 42% to about 100%, or even about 43% to about 100% of the cells in the composition. In some embodiments, the composition comprises either a population of MILS that do not express 4-1BB, e.g., as detected by flow cytometry, or a population of MILS that expresses low levels of 4-1BB, i.e., relative to the expression level of MILS from the population of MILS that express 4-1BB.

In some embodiments, the composition comprises MILS that express CD4.

In some embodiments, the composition comprises MILS that express CD8.

In some embodiments, the composition comprises MILS that express CD4. In some embodiments, the composition comprises MILS that express CD8. In some embodiments, the ratio of CD4⁺:CD8⁺ MILs present in the composition is about 2:1.

The composition may comprise a population of MILS that expresses CD8. The population of MILS that expresses CD8 may comprise a plurality of MILS that expresses CXCR4.

In some embodiments, the composition comprises a population of MILS that expresses CD4. The population of MILS that expresses CD4 may comprise a plurality of MILS that expresses CXCR4.

The MILS may express the different factors or surface receptors as described herein alone or in combination with one another. Thus, for example, a MIL can be CD3+, CD4+, and CD8+. Such cells can also express IFNγ. The cells can also be positive or negative for the various factors or receptors provided for herein.

In some embodiments, the methods for preventing or treating cancer in a subject are provided. In some embodiments, the methods comprise administering to a subject one of the compositions described herein, such as, but not limited to, cancer-specific MILS as provided herein. In some embodiments, the compositions are administered as provided for herein. In some embodiments, the method comprises administering to the subject a therapeutically effective amount of any one of the compositions described herein. In some embodiments, the method comprises administering to the subject a therapeutically-effective amount of the cancer specific MILS. In some embodiments, the MILS are activated. In some embodiments, the MILS are hypoxic activated as described herein and referenced herein. In some embodiments, the MILS are cultured under hypoxic conditions followed by normoxic conditions as described and referenced herein. In some embodiments, MILS are obtained or extracted from a bone marrow sample obtained from a subject having cancer. In some embodiments, the MILS are allogeneic to the subject being treated. In some embodiments, the methods comprise culturing a bone marrow sample from a subject with an anti-CD3 antibody and an anti-CD28 antibody in a hypoxic environment of about 1% to about 3% oxygen to produce activated marrow infiltrating lymphocytes; and (b) culturing the activated marrow infiltrating lymphocytes in a normoxic environment in the presence of IL-2 to produce the composition. The composition can be then be administered to the subject with cancer.

The MILS provided for herein can also be engineered to further express a chimeric antigen receptor, which can also be referred to as a “CAR.” Examples of such CARs are known in the art. The CAR can be used to further add additional antigen specificity to the cancer specific MILS.

### EXAMPLES

The following examples are illustrative, but not limiting, of the compositions and methods described herein. Other suitable modifications and adaptations known to those skilled in the art are within the scope of the following embodiments.

### Example 1: TCRβ CDR3 Sequencing and Analysis

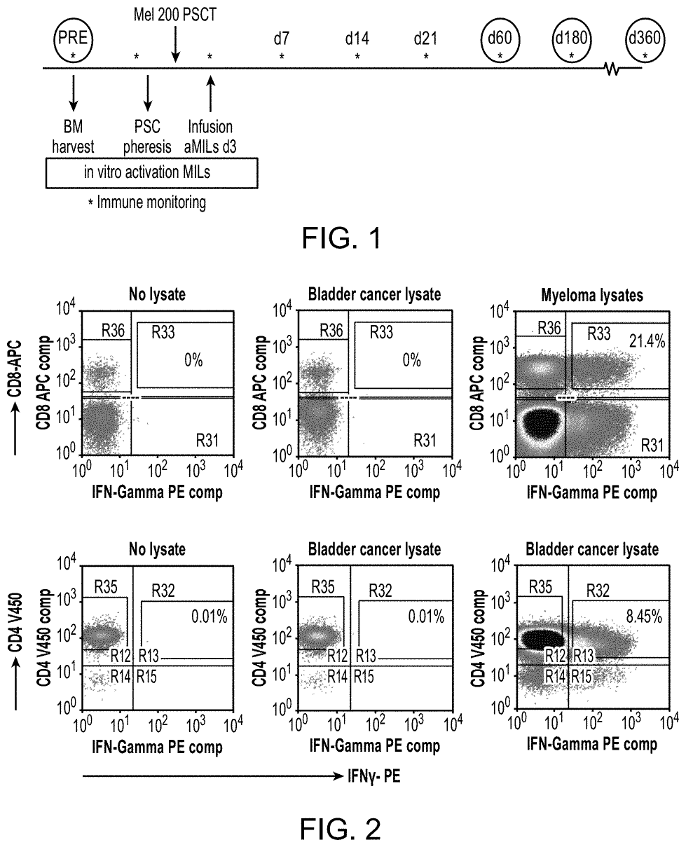

The TCRβ CDR3 was sequenced using Adaptive Biotechnologies' immunoSEQ Assay and used to identify and track MILS T cell clonotypes. The immunoSEQ assay was used on 11 specimens (unsorted MILS, IFNγ-capture-sorted tumor antigen-specific CD4+ and CD8+ T cells, and blood and BM collected pre-treatment and 60, 180 and 360 days post-infusion) from 6 patients (3 clinical responders who achieved a CR and 3 non-responders whose disease progressed) from the Phase I study (see FIG. 1).

### Example 2: IFNγ Capture Isolation of Tumor Antigen-specific MILS

MILS products were cultured with CFSE-labelled autologous BMMC pulsed with SW780 bladder cancer cell line lysate (bladder cancer lysate), a mixture of NIH929 and U266 multiple myeloma cell line lysates (Myeloma Lysates) or without tumor cell lystates (No Lysate). After 5 days, IFNγ-capture-based FACS was used to isolate IFNγ-producing CD8+ and CD4+ tumor antigen-specific T cells. FACS sorting data for a representative patient are shown in FIG. 2. The percentage of IFNγ-producing CD8+(top row, FIG. 2) and CD4+(bottom row, FIG. 2) T cells for each culturing condition are shown.

### Example 3: Clonality

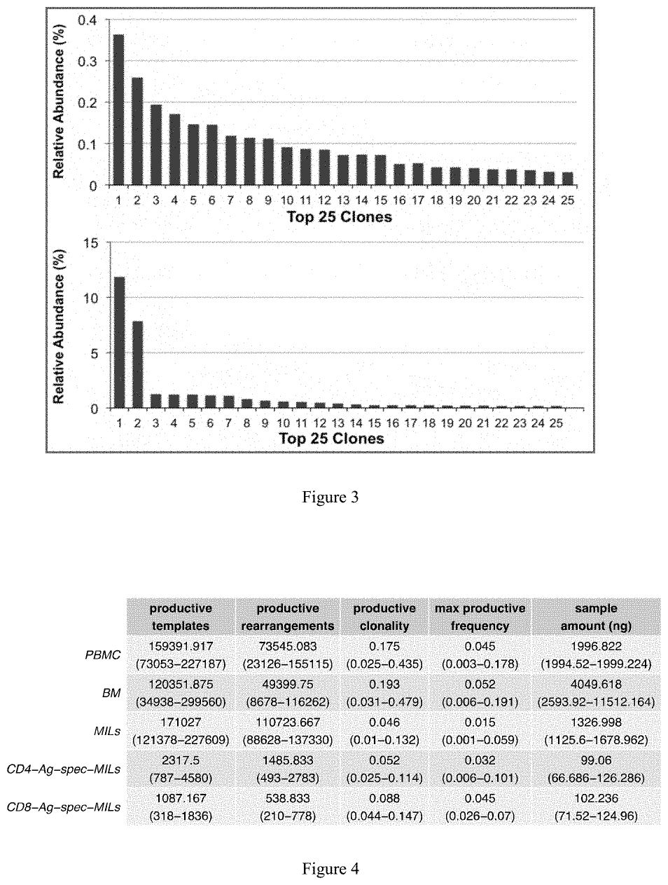

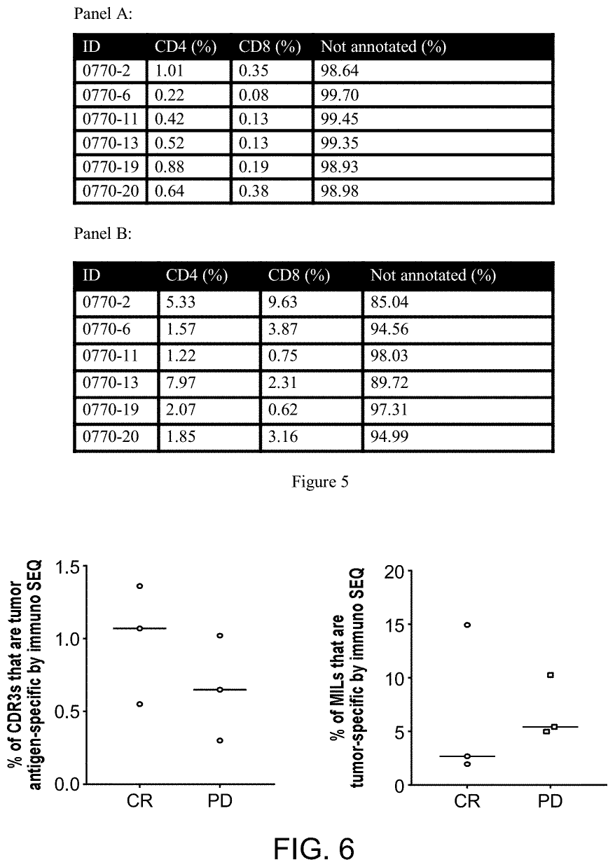

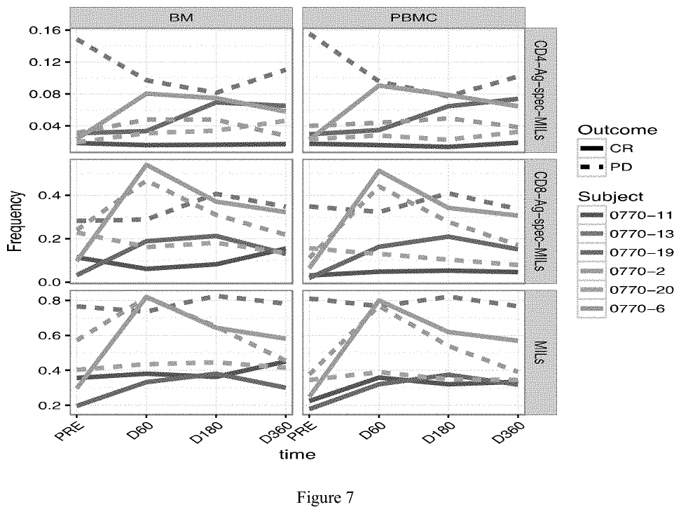

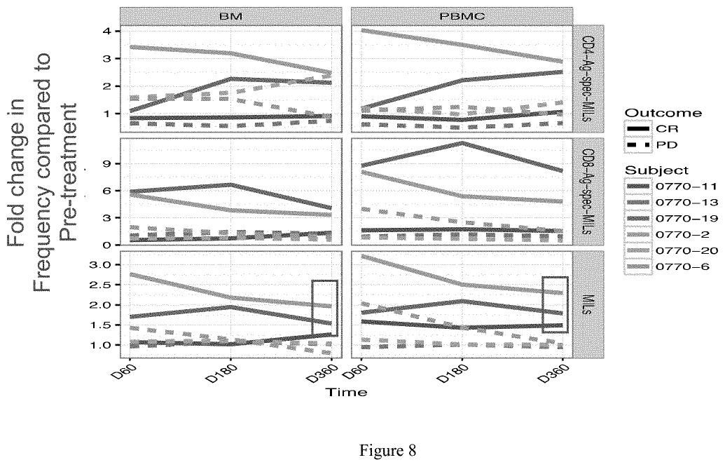

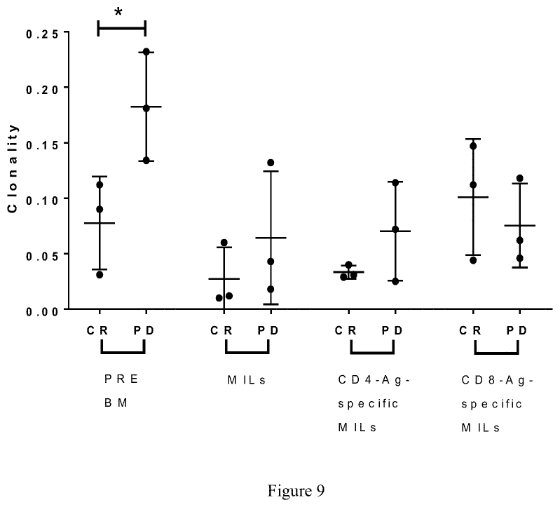

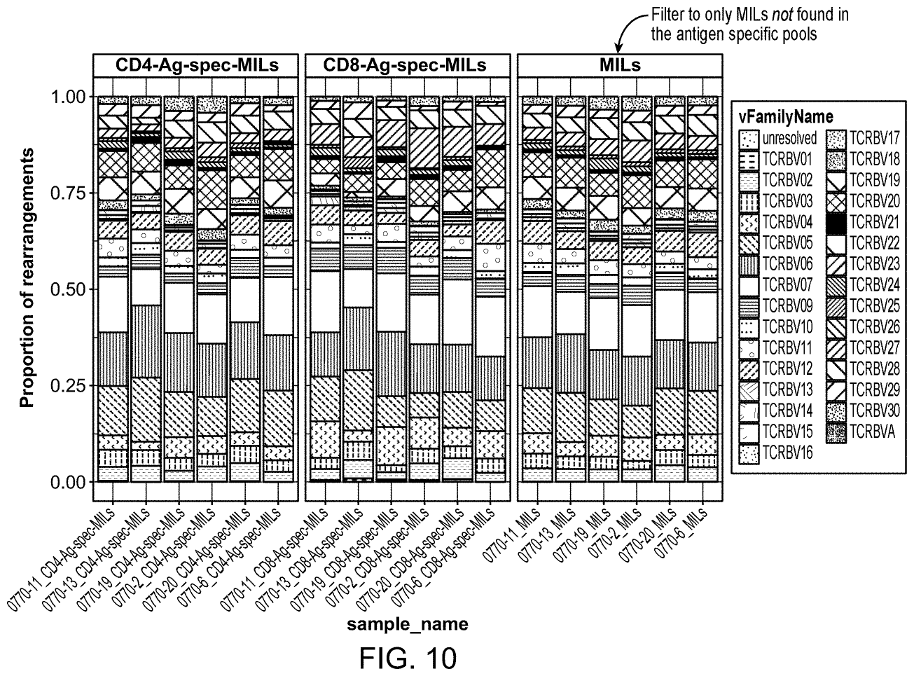

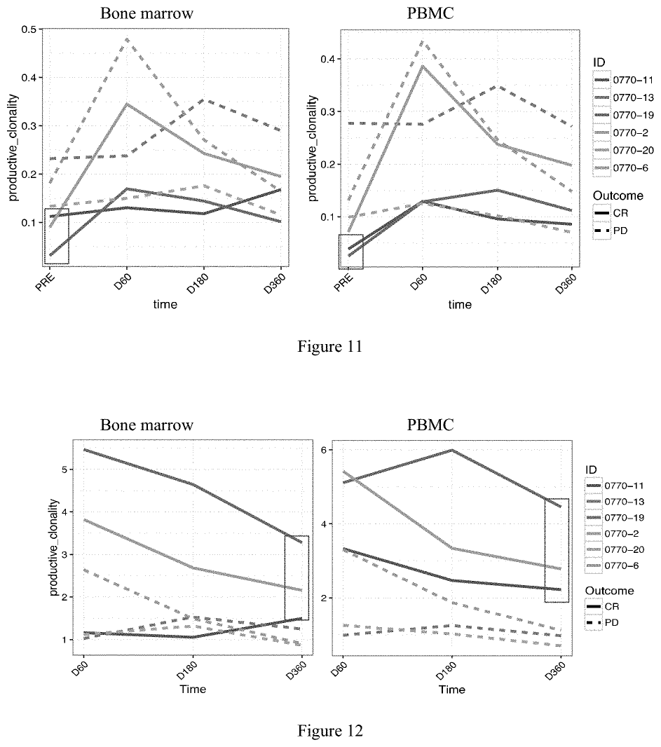

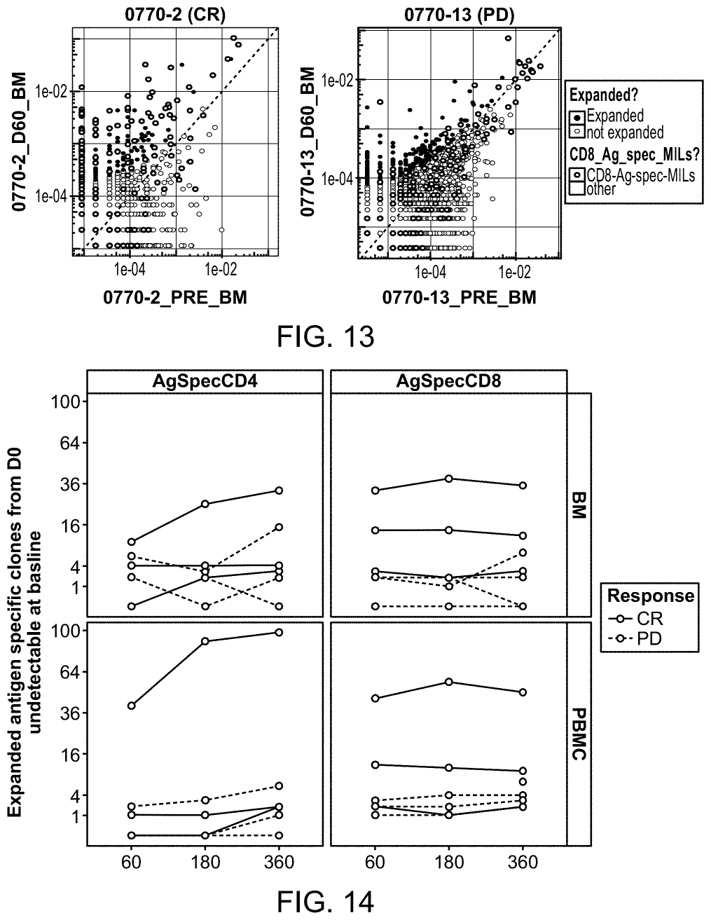

The extent of mono- or oligoclonal expansion was quantitated by measuring the shape of the clone frequency distribution (see FIG. 3). Values range from 0 to 1, where values approaching 1 indicate a nearly monoclonal population. Also, clonality equals 1 minus Pielou's evenness. The frequency distribution in FIG. 3 is calculated as follows:

$$Diversity = H = - {\sum }_{i = 1}^{N} {p}_{i} {log}_{2} ( {p}_{i} )$$

$$Clonality = 1 - \frac{H}{{log}_{2} ( N )} \\ !$$

In these equations, pᵢ is the proportional abundance of clone i, and N is the total number of unique receptor gene rearrangements.

When cumulative frequencies of MILS T-cell clonotypes were tracked in BM and blood, there were significant differences between responders and non-responders. Responders had a lower frequency of clonotypes at baseline but showed larger and more persistent increases in the frequency of clonotypes in both BM and blood. At day 360, fold-change from baseline in the frequency of MILS in both compartments segregated responders from non-responders.

In general, T-cell repertoires in MILS were highly polyclonal and no specific TCR TCRβ variable genes were enriched in tumor antigen-specific T cells suggesting that multiple antigens are targeted. In all 6 patients, MILS were more polyclonal than pre-expanded BM. However, starting repertoires were more polyclonal in responders, and responders had larger and more persistent post-infusion increases in clonality. At day 360, all 3 responders maintained an increase in clonality whereas clonality returned to baseline or lower in all 3 non-responders.

An increase of clonality in blood and bone marrow post-infusion correlates with therapeutic advantage. Therefore, this suggests that a composition of MILS that causes an increase in clonality measured in blood or bone marrow can provide a therapeutic advantage. The data also demonstrate the highly polyclonal nature of tumor antigen-specific T cells within MILS, which can provide an advantage against heterogeneous tumors.

In conclusion, the results provided herein provide evidence regarding the repertoire of T cell clonotypes in MILS and how the T cell repertoire evolves in blood and BM after treatment with MILS. They data also provides an estimate of the proportion of MILS that are tumor antigen-specific (2-15%). The data demonstrate the highly polyclonal nature of tumor antigen-specific T cells within MILS, which should provide an advantage against heterogeneous tumors. Treatment with MILS can induce the expansion of tumor antigen-specific T cells that are at very low frequencies and undetectable by immunoSEQ prior to treatment. The data demonstrate the potential for using the T cell repertoire to select and monitor patients treated with MILS. Lower clonality and lower frequencies of tumor antigen-specific T cells at baseline can also be used predict which patients are most likely to respond to treatment with MILS. The clonal expansion of MILS following infusion can be used to predict which patients are responding to treatment with MILS.

This description is not limited to the particular processes, compositions, or methodologies described, as these may vary. The terminology used in the description is for the purpose of describing the particular versions or embodiments only, and it is not intended to limit the scope of the embodiments described herein. Unless defined otherwise, all technical and scientific terms used herein have the same meanings as commonly understood by one of ordinary skill in the art. In some cases, terms with commonly understood meanings are defined herein for clarity and/or for ready reference, and the inclusion of such definitions herein should not necessarily be construed to represent a substantial difference over what is generally understood in the art. However, in case of conflict, the patent specification, including definitions, will prevail.

From the foregoing, it will be appreciated that various embodiments of the present disclosure have been described herein for purposes of illustration, and that various modification can be made without departing from the scope and spirit of the present disclosure. Accordingly, the various embodiments disclosed herein are not intended to be limiting.

What is claimed:

1. A composition comprising a population of marrow infiltrating lymphocytes comprising a plurality of TCRβs with affinities for a heterogeneous population of antigens.

2. A method for treating a subject having cancer with marrow infiltrating lymphocytes, the method comprising the steps of:
(a) culturing a bone marrow sample obtained from the subject having cancer with an anti-CD3 antibody and an anti-CD28 antibody in a hypoxic environment to produce hypoxic-activated marrow infiltrating lymphocytes;
(b) culturing the hypoxic-activated marrow infiltrating lymphocytes in a normoxic environment to produce the therapeutic activated marrow infiltrating lymphocytes comprising a plurality of TCRβs with affinities for a heterogeneous population of antigens; and
(c) administering the therapeutic activated marrow infiltrating lymphocytes to the subject having cancer.

3. The method of claim 2, wherein the hypoxic environment has an oxygen content of about 1% to about 3% oxygen.

4. The method of claim 2, wherein the lymphocytes are cultured in the presence of IL-2.

5. The method of claim 2, wherein the culturing the hypoxic-activated marrow infiltrating lymphocytes in a normoxic environment is performed in the presence of IL-2.

6. The method of claim 2, wherein the bone marrow sample is cultured in the hypoxic environment for about 24 hours.

7. The method of claim 2, wherein the bone marrow sample is cultured in the hypoxic environment for about 2 days.

8. The method of claim 2, wherein the bone marrow sample is cultured in the hypoxic environment for about 3 days.

9. The method of claim 2, wherein the bone marrow sample is cultured in the hypoxic environment for about 2 to about 5 days.

10. The method of claim 2, wherein the hypoxic environment is about 1% to about 2% oxygen.

11. The method of claim 2, wherein the hypoxic-activated marrow infiltrating lymphocytes are cultured in the normoxic environment for about 2 to about 12 days.

12. The method of claim 2, wherein the hypoxic-activated marrow infiltrating lymphocytes are cultured in the normoxic environment for about 6 days.

13. The method of claim 2, wherein the hypoxic-activated marrow infiltrating lymphocytes are cultured in the normoxic environment for about 9 days.

14. The method of claim 2, further comprising the step of removing a bone marrow sample from a subject having cancer prior to step (a).

15. The method of claim 2, wherein the anti-CD3 antibody and the anti-CD28 antibody are bound on a bead.

16. The method of claim 2, wherein the cancer is one or more of the cancers described herein.

17. The method of claim 2, wherein the cancer is selected from the group consisting of myeloma; lung cancer; prostate cancer; breast cancer; colon cancer; skin cancer; sarcomas and carcinomas selected from the group consisting of fibrosarcoma, myxosarcoma, liposarcoma, chondrosarcoma, and osteosarcoma; synovioma; mesothelioma; Ewing's tumor; leiomyosarcoma; rhabdomyosarcoma; lymphoid malignancy; pancreatic cancer; ovarian cancer; hepatocellular carcinoma; squamous cell carcinoma; basal cell carcinoma; adenocarcinoma; sweat gland carcinoma; medullary thyroid carcinoma; papillary thyroid carcinoma; pheochromocytomas sebaceous gland carcinoma; papillary carcinoma; papillary adenocarcinomas; medullary carcinoma; bronchogenic carcinoma; renal cell carcinoma; hepatoma; bile duct carcinoma; choriocarcinoma; Wilms' tumor; cervical cancer; testicular tumor; seminoma; bladder carcinoma; melanoma; CNS tumors selected from the group consisting of glioma, brainstem glioma, mixed gliomas, glioblastoma astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, and brain metastases, and combinations thereof.

18. The method of claim 2, wherein the cancer is a hematological or hematogenous cancer selected from the group consisting of leukemia, acute leukemias, acute lymphocytic leukemia, acute myelocytic leukemia, acute myelogenous leukemia, myeloblastic, promyelocytic, myelomonocytic, monocytic erythroleukemia, chronic leukemias, chronic myelocytic (granulocytic) leukemia, chronic myelogenous leukemia, and chronic lymphocytic leukemia, polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, myelodysplasia, and combinations thereof.

19. A method for treating a subject having cancer with therapeutic activated marrow infiltrating lymphocytes comprising a population of marrow infiltrating lymphocytes comprising a plurality of TCRβs with affinities for a heterogeneous population of antigens, the method comprising the steps of:
(a) culturing a bone marrow sample obtained from the subject having cancer with anti-CD3/anti-CD28 beads in a hypoxic environment of about 1% to about 2% oxygen for about 2 to about 5 days to produce hypoxic-activated marrow infiltrating lymphocytes comprising the population of marrow infiltrating lymphocytes comprising a plurality of TCRβs with affinities for a heterogeneous population of antigens;
(b) culturing the hypoxic-activated marrow infiltrating lymphocytes in a normoxic environment of about 21% oxygen for about 2 to about 12 days in the presence of IL-2 to produce the therapeutic activated marrow infiltrating lymphocytes; and
(c) administering the therapeutic activated marrow infiltrating lymphocytes to the subject having cancer.

20. A method of treating cancer in a subject, the method comprising administering a pharmaceutical composition comprising the composition of claim 1 to the subject.

21. The method of claim 20, wherein the population of marrow infiltrating lymphocytes is obtained from a subject having cancer.

22. The method of claim 20, wherein the cancer specific marrow infiltrating lymphocyte is autologous to the subject being treated.

23. The method of claim 20, wherein the cancer specific marrow infiltrating lymphocyte is allogeneic to the subject being treated.

24. The method of claim 20, wherein the marrow infiltrating lymphocyte is hypoxic activated.

25. The method of claim 20, wherein the marrow infiltrating lymphocyte is hypoxic activated and normoxic activated.

26. The method of claim 20, wherein the pharmaceutical composition is administered by parenteral administration, intraperitoneal or intramuscular administration.

27. The method of claim 2, wherein the about 75% to about 100% of marrow infiltrating lymphocytes administered to the subject express CD3.

28. The method of claim 2, wherein the about 80% to about 100% of marrow infiltrating lymphocytes administered to the subject express CD3.

29. The method of claim 2, wherein the about 85% to about 100% of marrow infiltrating lymphocytes administered to the subject express CD3.

30. The method of claim 2, wherein the about 90% to about 100% of marrow infiltrating lymphocytes administered to the subject express CD3.

31. The method of claim 2, wherein the ratio of CD4⁺:CD8⁺ T cells present in the composition or MILS administered to the subject is about 2:1.

32. A composition comprising a population of hypoxic-activated marrow infiltrating lymphocytes isolated from a patient with cancer comprising a population of marrow infiltrating lymphocytes comprising a plurality of TCRβs with affinities for a heterogeneous population of antigens, wherein about 75% to about 100% of the population of the hypoxic activated marrow infiltrating lymphocytes expresses CD3.

33. The composition of claim 32, wherein about 80% to about 100% of the population of the hypoxic activated marrow infiltrating lymphocytes expresses CD3.

34. The composition of claim 32, wherein about 85% to about 100% of the population of the hypoxic activated marrow infiltrating lymphocytes expresses CD3.

35. The composition of claim 32, wherein about 90% to about 100% of the population of the hypoxic activated marrow infiltrating lymphocytes expresses CD3.

36. The composition of claim 32, wherein the ratio of CD4⁺:CD8⁺ T cells present in the composition is about 2:1.

37. The composition of claim 32, wherein the cell population is obtainable from a bone marrow sample obtained from a subjecting having cancer by:
(a) culturing the bone marrow sample with an anti-CD3 antibody and an anti-CD28 antibody in a hypoxic environment of about 1% to about 3% oxygen to produce activated marrow infiltrating lymphocytes; and
(b) culturing the activated marrow infiltrating lymphocytes in a normoxic environment in the presence of IL-2 to produce the composition.

38. The composition of any of claim 32, wherein the MILS are cancer specific.

39. A method of predicting which patients are responding to infusion with MILS comprising:
A) isolating MILS from patients post-infusion;
B) determining the clonality of post-infusion isolated MILS;
C) continuing successful treatment with MILS if clonality is increased as compared to the clonality in pre-treatment bone marrow from the same patients.

40. A method of predicting which patients are responding to infusion with MILS comprising:
A) measuring clonality of baseline pre-infusion bone marrow;
B) measuring clonality of post-infusion bone marrow;
C) continuing successful treatment with MILS if clonality increases post-infusion as compared to pre-infusion bone marrow.

41. A method of predicting which patients are responding to infusion with MILS comprising:
A) measuring clonality of baseline pre-infusion blood;
B) measuring clonality of post-infusion blood;
C) continuing successful treatment with MILS if clonality increases post-infusion as compared to pre-infusion blood.
